(19) United States
(12) Patent Application Publication
(10) Pub. No.: US 2004/0186536 A1
(43) Pub. Date: Sep. 23, 2004

(54) Device with adjustable laser panels for stimulating hair growth

(76) Inventor: Donald J. Osendowski, Waukesna, WI (US)

Correspondence Address: Donald J. Ersler, 725 Garvens Avenue, Brookfield, WI, 53005, US

(21) Appl. No.: 10/391,937

(22) Filed: Mar. 19, 2003

(51) Int. Cl. A61N 1/00

(52) U.S. Cl. 607/89

(57) ABSTRACT

A laser device with adjustable panels for stimulating hair growth preferably includes a pedestal, an adjustable elbow and a plurality of laser panels. One end of the adjustable elbow is attached to the pedestal and the other end of the adjustable elbow is attached to a top laser panel. A first side laser panel is pivotally attached to a first side of the top laser panel and a second side laser panel is pivotally attached to a second side thereof. A rear laser panel is pivotally attached to a rear of the top laser panel. A plurality of laser modules are retained in each laser panel. A lens cap is preferably placed on the end of each laser module. The plurality of laser modules are preferably powered by a drive circuit.

### BACKGROUND OF THE INVENTION

1. Field of the Invention

The present invention relates generally to stimulating hair growth with lasers and more specifically to a device with adjustable laser panels for stimulating hair growth.

2. Discussion of the Prior Art

It appears that only one patent application exists for a stationary device which is capable of treating the scalp with a laser to stimulate hair growth. Patent application no. 2002/0188334 to Carlgren discloses a method and device stimulating the activity of hair follicles. The Calgren device includes a helmet with a plurality of diodes arranged therein. The helmet is placed over the head of a patient.

Accordingly, there is a clearly felt need in the art for a device with adjustable laser panels for stimulating hair growth that allows adjustability for patients with different shaped heads and scalps.

### SUMMARY OF THE INVENTION

The present invention provides a laser with adjustable laser panels for stimulating hair growth of a patient. The device with adjustable laser panels for stimulating hair growth (adjustable laser device) preferably includes a pedestal, an adjustable elbow and a plurality of laser panels. One end of the adjustable elbow is attached to the pedestal and the other end of the adjustable elbow is attached to a laser panel. A first side laser panel is pivotally attached to a first side of the top laser panel and a second side laser panel is pivotally attached to a second side of the top laser panel. A rear laser panel is pivotally attached to a rear of the top laser panel.

The adjustable elbow and plurality of laser panels do not require precise seating of the patient relative to the adjustable laser device. A plurality of laser modules are retained in each laser panel. A lens cap is preferably placed on the end of each laser module to disperse the laser light therefrom. The plurality of laser modules are preferably powered by a drive circuit. The drive circuit includes a DC power supply and a timer. The DC power supply inputs 115 volts AC and outputs a DC voltage. The timer is preferably connected in series between the DC power supply and the plurality of laser modules. The timer acts as an on-off switch, so that the scalp of the patient is exposed to the laser light for a limited amount of time.

Accordingly, it is an object of the present invention to provide an adjustable laser device for stimulating hair growth of patients with different shaped heads and scalps.

These and additional objects, advantages, features and benefits of the present invention will become apparent from the following specification.

### BRIEF DESCRIPTION OF THE DRAWINGS

FIG. 1 is a front view of an adjustable laser device in accordance with the present invention.

FIG. 2 is a side view of an adjustable laser device in accordance with the present invention.

FIG. 3 is a top view of an adjustable laser device in accordance with the present invention.

FIG. 4 is a perspective view of a patient receiving treatment from an adjustable laser device in accordance with the present invention.

FIG. 5 is a cross sectional view of a laser panel of an adjustable laser device in accordance with the present invention.

FIG. 6a is an enlarged side cross sectional view of a lens cap attached to an end of a laser module of an adjustable laser device in accordance with the present invention.

FIG. 6b is an enlarged top view of a lens cap attached to an end of a laser module of an adjustable laser device in accordance with the present invention.

FIG. 7 is an enlarged side view of a pivotal connection between a first side laser panel and a top laser panel of an adjustable laser device in accordance with the present invention.

FIG. 8 is a schematic diagram of a drive circuit of a control unit of an adjustable laser device in accordance with the present invention.

### DETAILED DESCRIPTION OF THE PREFERRED EMBODIMENTS

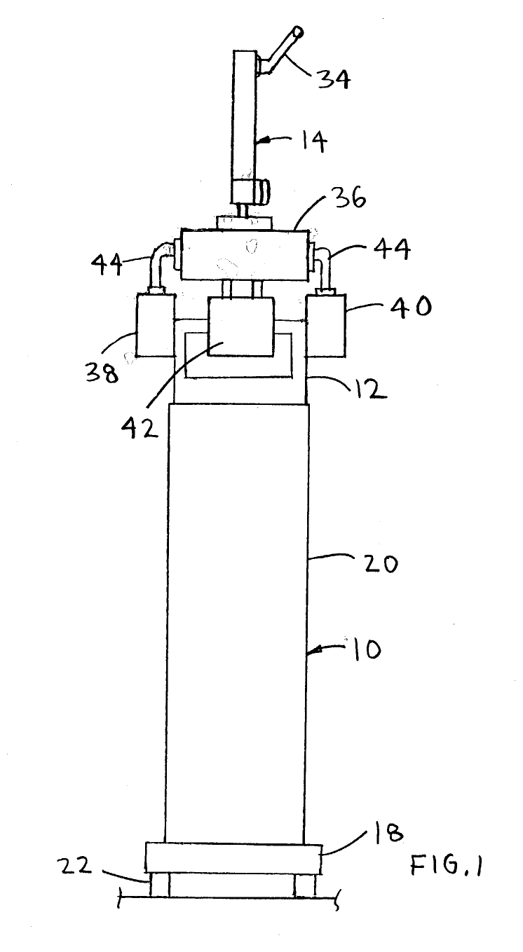

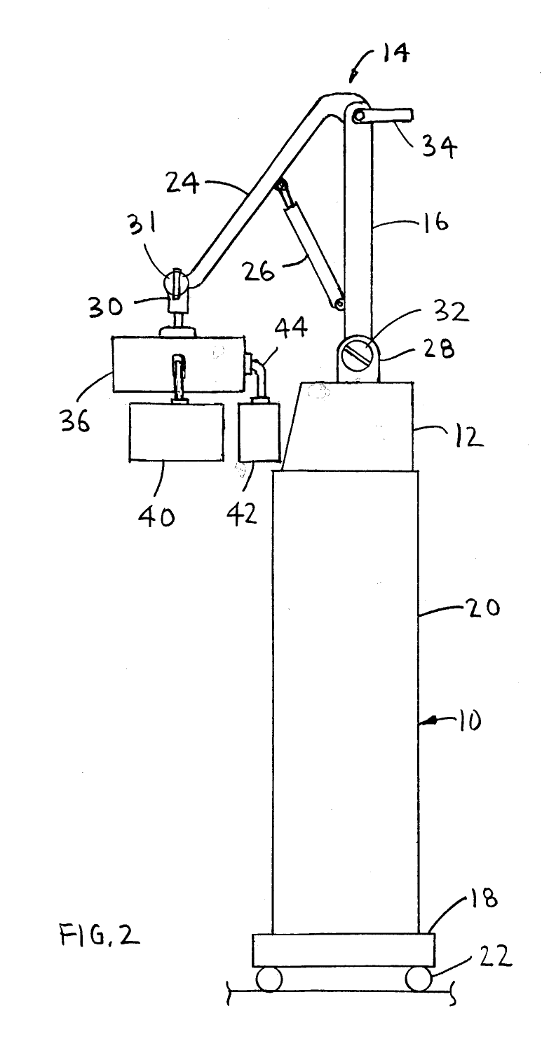

With reference now to the drawings, and particularly to FIG. 1, there is shown a front view of an adjustable laser device 1. With reference to FIGS. 2-3, the adjustable laser device 1 includes a pedestal 10, an adjustable elbow 14 and a plurality of laser panels. The pedestal 10 preferably includes a control unit 12, a base 18 and a column 20. The column 20 extends upward from a top of the base 18 and a set of wheels 22 are attached to a bottom of the base 18. The control unit 12 is attached to a top of the column 20. With reference to FIG. 8, a drive circuit 24 is retained in the control unit 12. Control devices such as switches and displays are preferably disposed on a front of the control unit 12. The adjustable elbow 14 preferably includes a first leg 16, a second leg 24, a support strut 26, a first pivotal base 28 and a second pivotal attachment member 30.

The first pivotal base 28 is attached to a top of the control unit 12. One end of the first leg 16 is pivotally retained by the first pivotal base 28. The angular position of the first leg 16 relative to the first pivotal base 28 is preferably secured by tightening a first knob 32. The other end of the first leg 16 is pivotally connected to one end of the second leg 24. The angular position of the first leg 16 relative to the second leg 24 is preferably secured by tightening a first handle 34. One end of the support strut 26 is pivotally attached to the first leg 16 and the other end is pivotally attached to the second leg 24. The support strut 26 supports the weight of the plurality of laser panels. The second pivotal member 30 is attached to the other end of the second leg 24. The angular position of the second leg 24 relative to the second pivotal member 30 is preferably secured by tightening a second tightening knob 31.

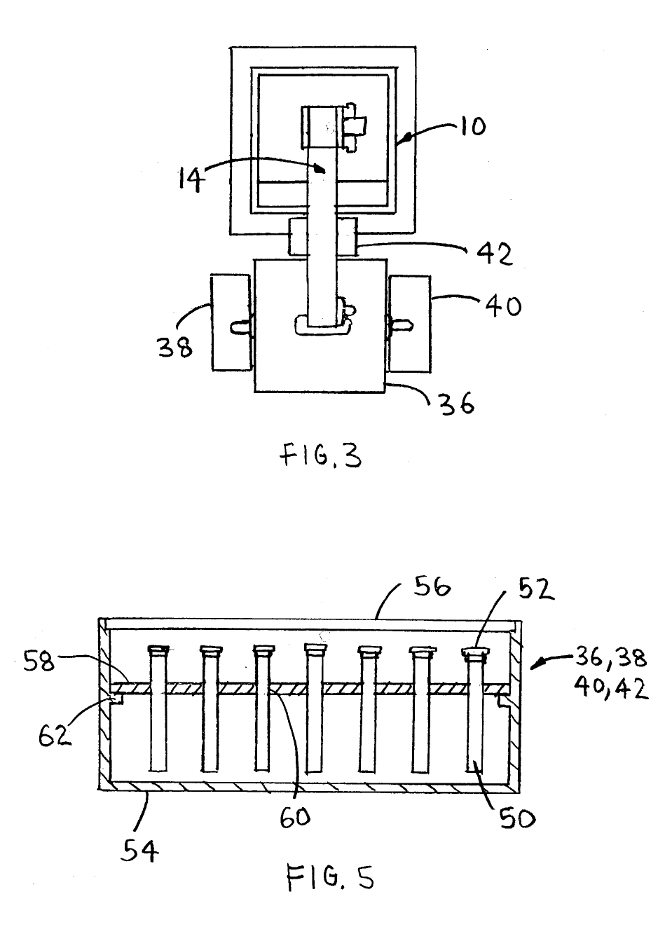

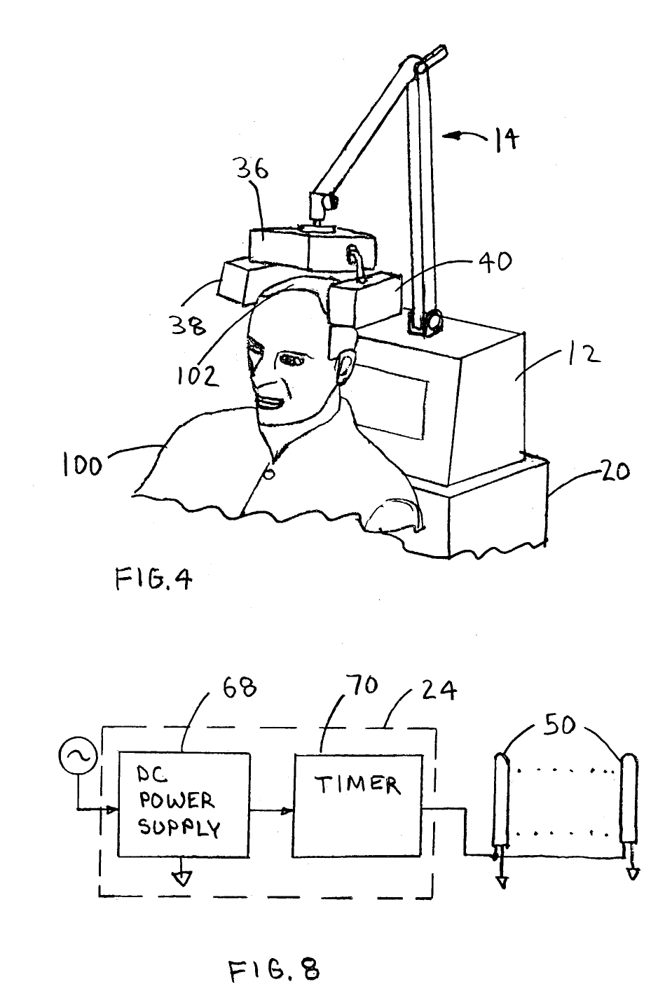

With reference to FIG. 4, the plurality of laser panels surround a scalp 102 of a patient 100. The plurality of laser panels include a top laser panel 36, a first side laser panel 38, a second side laser panel 40 and a rear laser panel 42. With reference to FIG. 7, the first side laser panel 38 is pivotally attached to the top laser panel 36, preferably with an elbow joint 44. A first leg 46 of the elbow joint 44 may be threaded into the first side laser panel 38 and the second leg 48 may be attached to the top laser panel 36 with fasteners. The pivotal connection between the first and second legs preferably requires a reasonable amount of force to change the angular orientation therebetween. The second side laser panel 38 and the rear laser panel 42 are also pivotally attached to the top laser panel 36 with the elbow joint 44. However, other devices may be used to secure the first side laser panel 38, the second side laser panel 40 and the rear laser panel 42 to the top laser panel 36, besides the elbow joint 44.

The adjustable elbow 14 and plurality of laser panels do not require precise seating of the patient relative to the adjustable laser device 1. With reference to FIG. 5, each laser panel 36, 38, 40, 42 includes a plurality of laser modules 50, a plurality of lens caps 52, an open box 54, a cover 56 and a retaining plate 58. A plurality of holes 60 are formed through the retaining plate 58 to receive the plurality of laser modules 50. The plurality of laser modules 50 are preferably secured in the plurality of holes 60 with adhesive or any other suitable retention process.

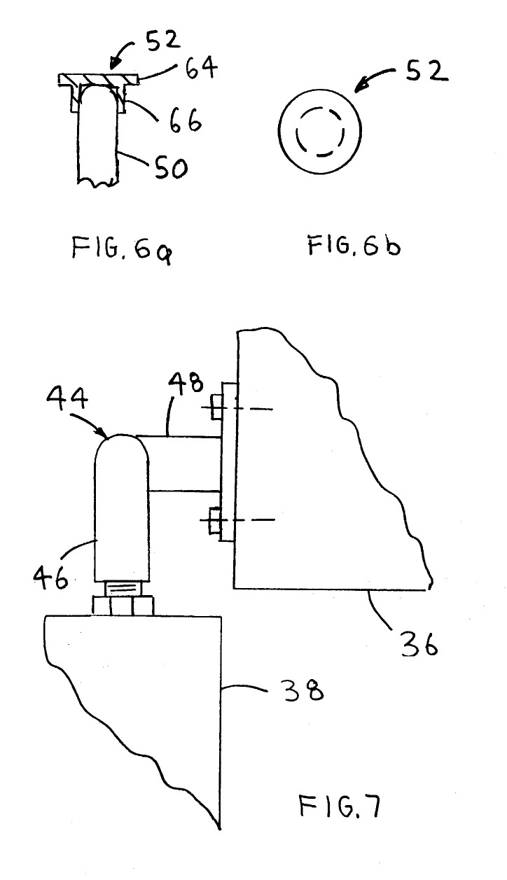

With reference to FIGS. 6a and 6b, a single lens cap 52 is secured to a top of each laser module 50. The lens cap 52 includes a flat base 64 and a flange 66 extending from one end thereof. The flange 66 is sized to firmly receive an end of the laser module 50. The lens cap 52 disperses laser light from the laser module over a wider area such that the scalp of a patient is fully exposed to the laser light. The open box 54 preferably includes a shelf 62, which the retaining plate 58 may be secured to with adhesive or any other suitable fastening method. The cover 56 is attached to a top of the open box 54 with any suitable method. The cover 56 is preferably fabricated from a clear plastic material.

With reference to FIG. 8, the plurality of laser modules 50 are preferably connected in parallel to an output of the drive circuit 24. The drive circuit 24 includes a DC power supply 68 and a timer 70. The DC power supply 68 inputs 115 volts AC and outputs a DC voltage. The timer 70 is connected in series between the DC power supply 68 and the plurality of laser modules 50. The timer 70 acts as an on-off switch, so that the scalp of the patient is exposed to the laser light for a limited amount of time. The timer 70 may be set to power the plurality of laser modules 50 for a set period of time. Further explanation of the DC power supply 68 and timer 70 is not required, since they are devices, which are well known in the art. Wires from the control circuit 24 to the top laser panel 36 are preferably routed through a center of the adjustable elbow 14 to the top laser panel 36. Wires are also routed from the top laser panel 36 to the first side laser panel 38, the second side laser panel 40 and the rear laser panel 42.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

I claim:

1. An adjustable laser device comprising:
a pedestal;
a drive circuit;
an adjustable elbow having a first end and a second end, a first end of said adjustable elbow being attached to said pedestal;
a top laser panel including a plurality of laser modules, said second end of said adjustable elbow being attached to said top laser panel;
a first side laser panel including a plurality of laser modules, said first side laser panel being pivotally connected to said top laser panel;
a second side laser panel including a plurality of laser modules, said second side laser panel being pivotally connected to said top laser panel; and
a rear laser panel including a plurality of laser modules, said rear laser panel being pivotally connected to said top laser panel, said drive circuit supplying electrical current to said plurality of laser modules.

2. The adjustable laser device of claim 1, further comprising:
said drive circuit including a DC power supply and a timer, said DC power supply supplying electrical current through said timer to said plurality of laser modules for a set period of time.

3. The adjustable laser device of claim 1, further comprising:
said adjustable elbow including a first leg, a second leg, a first pivotal base and a second pivotal attachment member, one end of said first leg being pivotal retained by said first pivotal base and the other end of said first leg is pivotal connected to one end of the second leg, the other end of the second leg is pivotally retained by the second pivotal attachment member.

4. The adjustable laser device of claim 1, further comprising:
each said laser panel including an open box, a cover, a retaining plate and said plurality of laser modules, said retaining plate having a plurality of holes to receive said plurality of laser modules, said retaining plate being retained by said open box, said cover being attached to a top of said open box.

5. The adjustable laser device of claim 1, further comprising:
a set of wheels being attached to a bottom of said pedestal.

6. The adjustable laser device of claim 1, further comprising:
said first side laser panel, said second side laser panel and said rear laser panel being pivotally attached to said top laser panel with at least three elbow joints.

7. The adjustable laser device of claim 1, further comprising:
a lens cap being attached to a top of each one of said plurality of laser modules.

8. An adjustable laser device comprising:
a pedestal;
a drive circuit having a DC power supply and a timer;
an adjustable elbow having a first end and a second end, a first end of said adjustable elbow being attached to said control unit;
a top laser panel including a plurality of laser modules, said second end of said adjustable elbow being attached to said top laser panel;
a first side laser panel including a plurality of laser modules, said first side laser panel being pivotally connected to said top laser panel;
a second side laser panel including a plurality of laser modules, said second side laser panel being pivotally connected to said top laser panel; and
a rear laser panel including a plurality of laser modules, said rear laser panel being pivotally connected to said top laser panel, said DC power supply supplying electrical current through said timer to said plurality of laser modules for a set period of time.

9. The adjustable laser device of claim 8, further comprising:
said adjustable elbow including a first leg, a second leg, a first pivotal base and a second pivotal attachment member, one end of said first leg being pivotal retained by said first pivotal base and the other end of said first leg is pivotal connected to one end of the second leg, the other end of the second leg is pivotally retained by the second pivotal attachment member.

10. The adjustable laser device of claim 8, further comprising:
each said laser panel including an open box, a cover, a retaining plate and said plurality of laser modules, said retaining plate having a plurality of holes to receive said plurality of laser modules, said retaining plate being retained by said open box, said cover being attached to a top of said open box.

11. The adjustable laser device of claim 8, further comprising:
a set of wheels being attached to a bottom of said pedestal.

12. The adjustable laser device of claim 8, further comprising:
said first side laser panel, said second side laser panel and said rear laser panel being pivotally attached to said top laser panel with at least three elbow joints.

13. The adjustable laser device of claim 8, further comprising:
a lens cap being attached to a top of each one of said plurality of laser modules.

14. A method of utilizing a plurality of laser modules to stimulate hair growth, comprising the steps of:
positioning a top laser panel having a plurality of laser modules on top of a patient's scalp;
positioning a first side laser panel having a plurality of laser modules on a first side of the patient's scalp, said first side laser panel being pivotally adjustable relative to said top laser panel;
positioning a second side laser panel having a plurality of laser modules on a second side of the patient's scalp, said second side laser panel being pivotally adjustable relative to said top laser panel;
positioning a rear laser panel having a plurality of laser modules on a rear of the patient's scalp, said rear laser panel being pivotally adjustable to said top laser panel; and
supplying said plurality of laser modules with electrical current for a set period of time.

15. The method of utilizing a plurality of laser modules to stimulate hair growth of claim 14, further comprising the step of:
providing a pedestal for suspending said laser panels.

16. The method of utilizing a plurality of laser modules to stimulate hair growth of claim 15, further comprising the step of:
providing an adjustable elbow having a first end and a second end, a first end of said adjustable elbow being attached to said pedestal and the other end of said pedestal being attached to said top laser panel.

17. The method of utilizing a plurality of laser modules to stimulate hair growth of claim 16, further comprising the step of:
providing said adjustable elbow with a first leg, a second leg, a first pivotal base and a second pivotal attachment member, one end of said first leg being pivotal retained by said first pivotal base and the other end of said first leg is pivotal connected to one end of the second leg, the other end of the second leg is pivotally retained by the second pivotal attachment member.

18. The method of utilizing a plurality of laser modules to stimulate hair growth of claim 14, further comprising the step of:
providing each said laser panel with an open box, a cover, a retaining plate and said plurality of laser modules, said retaining plate having a plurality of holes to receive said plurality of laser modules, said retaining plate being retained by said open box, said cover being attached to a top of said open box.

19. The method of utilizing a plurality of laser modules to stimulate hair growth of claim 15, further comprising the step of:
attaching a set of wheels to a bottom of said pedestal.

20. The method of utilizing a plurality of laser modules to stimulate hair growth of claim 14, further comprising the step of:
attaching a lens cap to a top of each one of said plurality of laser modules.
